(19) United States
(12) Patent Application Publication
(10) Pub. No.: US 2026/0347779 A1
(43) Pub. Date: Nov. 5, 2026

(54) METHOD FOR ACTIVATING PLATELETS TO RELEASE GROWTH FACTORS AND EXOSOMES, ELECTRONIC DEVICE, AND STORAGE MEDIUM

(71) Applicant: LifeTechX Inc., San Jose, CA (US)

(72) Inventors: YIJIE YANG, Shenzhen (CN); Haiyun Chen, Shenzhen (CN); Yuan Xiang, Shenzhen (CN); Jinghua Luo, Shenzhen (CN); Chuan Lai, Shenzhen (CN); Wan Wang, Shenzhen (CN)

(21) Appl. No.: 19/710,607

(22) Filed: Jun. 17, 2026

(30) Foreign Application Priority Data
Jan. 6, 2026 (CN) 202610013746.9

(51) Int. Cl. C12N 5/078; C12M 1/00; C12M 1/34; C12M 1/42; C12N 13/00

(52) U.S. Cl. CPC C12N 5/0644; C12M 31/00; C12M 35/04; C12M 41/12; C12N 13/00

(57) ABSTRACT

A method for activating platelets to release growth factors and exosomes is provided. The method includes determining a target activation mode for platelet-rich plasma based on a target usage of the platelet-rich plasma; activating at least one of a light-emitting device, an ultrasonic device, and a temperature control device of the electronic device based on the target activation mode; when the light-emitting device is activated, irradiating the platelet-rich plasma based on at least one preset light parameter; when the ultrasonic device is activated, ultrasonically treating the platelet-rich plasma based on at least one preset ultrasonic parameter; and when the temperature control device is activated, adjusting a temperature of an environment where the platelet-rich plasma is located based on at least one preset temperature control parameter, and enabling platelets to be activated to release growth factors and exosomes.

### FIELD

The subject matter herein generally relates to a field of plasma processing, and particularly to a method for activating platelets to release growth factors and exosomes, an electronic device, and a storage medium.

### BACKGROUND

Platelet-rich plasma (PRP) is a platelet concentrate obtained from autologous whole blood via centrifugation, it has a high platelet concentration and contains multiple growth factors, including platelet-derived growth factor (PDGF) and transforming growth factor-beta (TGF-β).

Before use, platelet-rich plasma is usually activated ex vivo through exogenous means to enable a rapid release of growth factors. Exogenous activation ensures that nearly all platelets undergo degranulation prior to injection, facilitating the delivery of high concentrations of growth factors to injured sites in the human body and avoiding the uncertainties associated with in vivo activation.

Exogenous activation of platelets is primarily achieved through chemical methods, which trigger the platelet activation process by adding chemical reagents. However, the chemical reagents used in these methods may have adverse effects on the human body, thereby impacting human health.

### BRIEF DESCRIPTION OF THE DRAWINGS

Many aspects of the disclosure can be better understood with reference to the following drawings. The components in the drawings are not necessarily drawn to scale, the emphasis instead being placed upon clearly illustrating the principles of the disclosure. Moreover, in the drawings, like reference numerals designate corresponding parts throughout the several views.

FIG. 1 illustrates a schematic view of an embodiment of an application scenario of a method for activating platelets to release growth factors and exosomes according to the present disclosure.

FIG. 2 illustrates a flowchart of an embodiment of the method for activating platelets to release growth factors and exosomes according to the present disclosure.

FIG. 3 illustrates a schematic view of an embodiment of target activation method mode determination according to the present disclosure.

FIG. 4 illustrates a schematic view of an embodiment of parameter control corresponding to a target activation mode according to the present disclosure.

FIG. 5 is a block diagram of an embodiment of an electronic device according to the present disclosure.

### DETAILED DESCRIPTION

Multiple embodiments are described in the present disclosure, but the description is exemplary rather than limiting, and there may be more embodiments and implementation solutions within the scope of the embodiments described in the present disclosure. Although many possible combinations of features are shown in the drawings and discussed in the detailed description, many other combinations of the disclosed features are also possible. Unless specifically limited, any feature or element of any embodiment may be used in combination with or in place of any other feature or element of any other embodiment.

When describing representative embodiments, the specification may present methods and/or processes as a specific sequence of steps. However, to the extent that the method or process does not depend on the specific order of steps described in the present disclosure, the method or process should not be limited to the specific order of steps described. As understood by those of ordinary skills in the art, other orders of steps are also possible. Therefore, the specific order of steps set forth in the specification should not be interpreted as limitation to the claims. In addition, the claims for the method and/or process should not be limited to the steps performed in the written order, and those skilled in the art may readily understand that these orders may vary and still remain within the essence and scope of the embodiments of the present disclosure.

Unless otherwise defined, technical terms or scientific terms used in the embodiments shall have common meanings as construed by those of ordinary skills in the art to which the present disclosure pertains. The terms “first”, “second” and the like used in the embodiments of the present disclosure do not represent any order, quantity, or importance, but are merely used to distinguish different components. The terms “include”, “contain” or the like mean that elements or articles so characterized do not exclude other elements or articles. The terms “connect”, “link” or the like are not limited to physical or mechanical connection, but may include electrical connections, whether direct or indirect.

Platelet-rich plasma (PRP) is a platelet concentrate obtained from autologous whole blood via centrifugation, its platelet concentration can reach 4-8 times that of normal blood, and it contains various growth factors such as PDGF and TGF-β.

Before use, platelet-rich plasma is activated ex vivo through chemical or physical methods, which is referred to as exogenous activation. Platelet activation leads to the rapid release of growth factors, and most growth factors can be released. Exogenous activation ensures that nearly all platelets undergo degranulation prior to injection, allowing a high concentration of growth factors to be delivered directly to the injured site in the human body in a single application, avoiding the uncertainties associated with in vivo activation.

Exogenous activation of platelets is primarily achieved through chemical methods, which trigger the platelet activation process by adding chemical reagents such as calcium chloride and thrombin. However, the chemical reagents or preservatives used in these methods may have adverse effects on the human body, thereby impacting human health.

To address the above-mentioned problem that exogenous activation of platelets is primarily achieved through chemical methods, and the chemical reagents used in these methods may have adverse effects on the human body, potentially impacting human health, the embodiments of the present disclosure provide a method for activating platelets to release growth factors and exosomes. The method can achieve exogenous activation of platelets based on a physical method combining ultrasound, light, and temperature control, allowing platelets to release exosomes while also releasing growth factors, without introducing chemical reagents, thus offering high safety and effectively protecting human health.

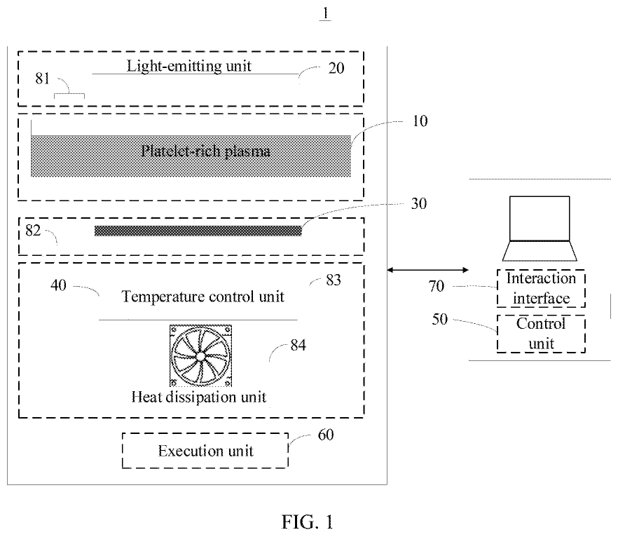

FIG. 1 illustrates a schematic view of an application scenario of a method for activating platelets to release growth factors and exosomes. An embodiment of the present disclosure provides an electronic device 1 for exogenous activation of platelets to release growth factors and exosomes. The electronic device 1 includes, but is not limited to: a container 10, a light-emitting device 20, an ultrasonic device 30, a temperature control device 40, a control device 50, an execution device 60, and an interaction interface 70.

An end of the container 10 is open, and the container 10 is used for holding platelet-rich plasma. The light-emitting device 20 may be a lamp device. The light-emitting device 20 is arranged above the container 10 and is used to provide a light source for irradiating the platelet-rich plasma. The ultrasonic device 30 is arranged at or below the bottom of the container 10 and is used to generate ultrasonic waves that act on the platelet-rich plasma. For example, the ultrasonic device 30 may be an ultrasonic generator. The temperature control device 40 is arranged below the container 10 or at another location close to the container 10, and is used to adjust the temperature of the environment where the platelet-rich plasma is located. For example, the temperature control device 40 may be an air conditioner, a thermal cycler, or the like.

The control device 50 is configured to determine a target activation mode for the platelet-rich plasma based on a target usage of the platelet-rich plasma. The target activation mode is determined from a number of preset activation modes. The number of preset activation modes include, but are not limited to, a light activation mode, an ultrasonic activation mode, a temperature control activation mode, a light and ultrasonic activation mode, a light and temperature control activation mode, an ultrasonic and temperature control activation mode, and a light, ultrasonic, and temperature control activation mode.

The execution device 60 is configured to activate at least one of the light-emitting device 20, the ultrasonic device 30, and the temperature control device 40 based on the target activation mode, and process the platelet-rich plasma, such that platelets are activated to release growth factors and exosomes. For example, the growth factors may be PDGF, TGF-β, etc., and the exosomes may include proteins, nucleic acids, lipids, etc. In one embodiment, the control device 50 and the execution device 60 may be processors.

The interaction interface 70 is configured to receive a user input of the target usage of the platelet-rich plasma. For example, the interaction interface 70 may be a touch screen.

The electronic device 1 may further include a memory (not shown in FIG. 1). The memory stores the number of preset activation modes and a relationship between a number of usages and a number of preset activation modes. The control device 50 determines a preset activation mode corresponding to the target usage based on the relationship between a number of usages and a number of preset activation modes and the user-input target usage of the platelet-rich plasma, and determines the preset activation mode corresponding to the target usage as the target activation mode.

In another embodiment, the control device 50 further sends the target activation mode to the interaction interface 70, and the interaction interface 70 displays the target activation mode for a confirmation input on the interaction interface, when the confirmation of the target activation mode is not received, a preset activation mode of the plurality of preset activation modes input by the user is received, the preset activation mode input by the user is determined as the target activation mode. For example, the target activation mode is displayed in a pop-up window, and the pop-up window includes options “Yes” and “No”. If the user triggers the option “No”, i.e., does not select the target activation mode, then a preset activation mode input by the user is received. For example, after the user triggers the option “No”, the number of preset activation modes are displayed in a list form, each preset activation mode corresponding to an option, and the preset activation mode selected by the user is determined based on the option triggered by the user. The interaction interface 70 sends the preset activation mode input by the user to the control device 50, and the control device 50 is further configured to determine the preset activation mode input by the user as the target activation mode. If the user triggers the option “Yes”, i.e., selects the target activation mode, then the preset activation mode corresponding to the target usage is determined as the target activation mode.

In one embodiment, the electronic device 1 further includes a light sensor 81, an ultrasonic amplitude feedback device 82, and a temperature sensor 83. The light sensor 81 is used to sense the light intensity of the light-emitting device. The ultrasonic amplitude feedback device 82 is used to detect the amplitude of the ultrasonic waves generated by the ultrasonic device 30. The ultrasonic amplitude feedback device 82 may be an ultrasonic sensor. The temperature sensor 83 is used to sense the ambient temperature of the environment where the platelet-rich plasma is located.

In one embodiment, the execution device 60 controls the light parameters of the light-emitting device based on the light intensity sensed by the light sensor, controls the temperature control parameters of the temperature control device based on the temperature sensed by the temperature sensor, and controls the ultrasonic device to generate ultrasonic waves based on preset ultrasonic parameters.

It can be understood that, in another embodiment, the electronic device 1 may also include a container 10, a light-emitting device 20, an ultrasonic device 30, a temperature control device 40, and an execution device 60, for performing exogenous activation of platelets based on a target activation mode. The control device 50 and the interaction interface 70 are provided by another electronic device, for interacting with the user to determine the target usage of the platelet-rich plasma and the target activation mode, and sending the target activation mode to the electronic device 1.

In one embodiment, the electronic device 1 may further include a heat dissipation unit 84, which is used to dissipate heat from the electronic device 1, or lowering the ambient temperature of the environment where the platelet-rich plasma is located. For example, the heat dissipation unit 84 may be a cooling fan.

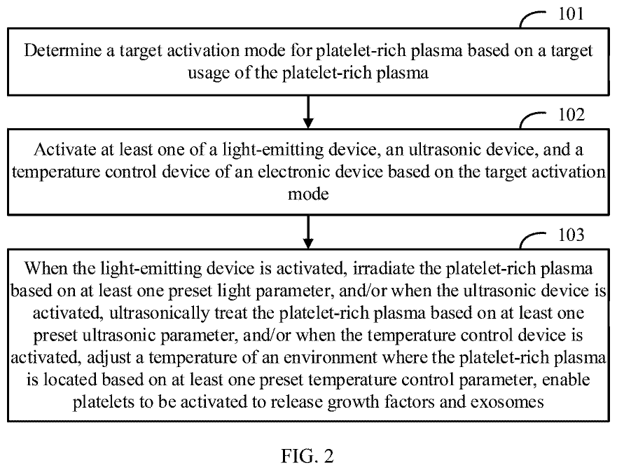

FIG. 2 illustrates a flowchart of a method for activating platelets to release growth factors and exosomes. The method is applied to the electronic device 1 in the above-described embodiments of the present disclosure. The method is provided by way of example, as there are a variety of ways to carry out the method. Each block shown in FIG. 2 represents one or more processes, methods, or subroutines carried out in the example method. Furthermore, the illustrated order of blocks is by example only and the order of the blocks can be changed. Additional blocks may be added or fewer blocks may be utilized, without departing from this disclosure. The example method can begin at block 101.

At block 101, determine a target activation mode for platelet-rich plasma based on a target usage of the platelet-rich plasma.

In one embodiment, the target usage of the platelet-rich plasma input by a user is received via an interaction interface, and the target activation mode for the platelet-rich plasma is determined by a control device based on the target usage.

In detail, the control device determines a preset activation mode of the number of preset activation modes corresponding to the target usage based on the relationship between the number of usages and the number of preset activation modes and the target usage, and determines the preset activation mode corresponding to the target usage as the target activation mode.

In another embodiment, the control device further sends the target activation mode to the interaction interface of the electronic device, displays the target activation mode via the interaction interface for a confirmation input on the interaction interface, and if the confirmation of the target activation mode is not received, for example, the user does not select the target activation mode, receives a preset activation mode input by the user, and the control device determines the preset activation mode input by the user as the target activation mode.

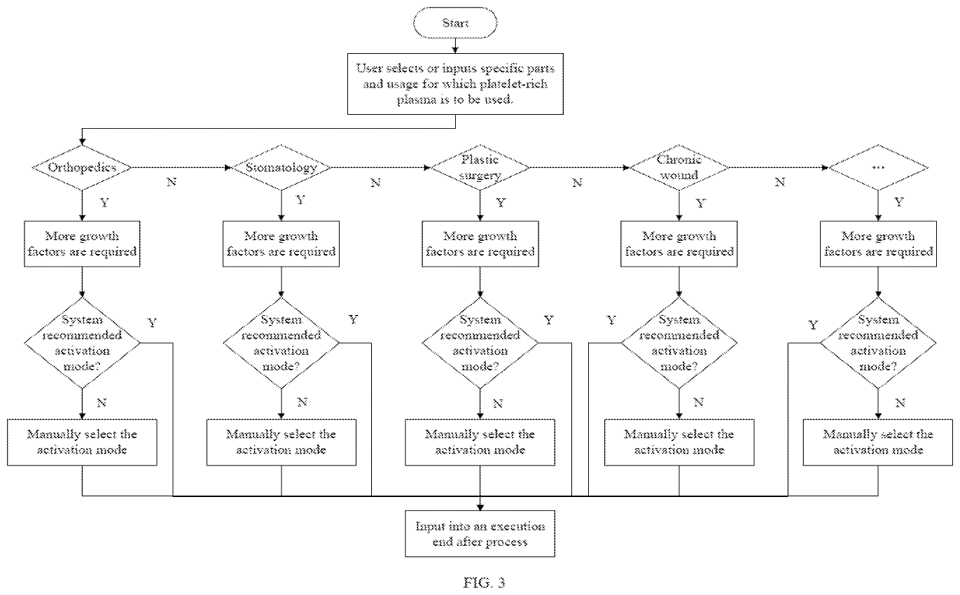

FIG. 3 illustrates a schematic view of a target activation mode determination. The target usage may be a clinical department requiring platelet-rich plasma or a part of the human body on which the platelet-rich plasma is to act. For example, the target usage may be orthopedics, dentistry, plastic surgery, chronic wounds, etc. After determining the target usage of the platelet-rich plasma based on a selection operation of the user or input operation, an activation requirement for platelets is selected based on the selection operation of the user, for example, a requirement to activate more growth factors. Based on the activation requirement, it is determined to trigger exogenous activation. Based on the selection operation of the user, it is determined whether to apply a system-recommended activation mode (i.e., the target activation mode). If the system-recommended activation mode is applied, the system-recommended activation mode is sent to the execution device. If the system-recommended activation mode is not used, an activation mode is determined based on the selection operation of the user, and the activation mode manually selected by the user is sent to the execution device.

At block 102, activate at least one of a light-emitting device, an ultrasonic device, and a temperature control device of an electronic device based on the target activation mode.

In one embodiment, if the target activation mode is a light activation mode, the temperature control device and the light-emitting device are activated. If the target activation mode is an ultrasonic activation mode, the temperature control device and the ultrasonic device are activated. If the target activation mode is a temperature control activation mode, the temperature control device is activated. If the target activation mode is a light and ultrasonic activation mode, the temperature control device, the light-emitting device, and the ultrasonic device are activated. If the target activation mode is a light and temperature control activation mode, the temperature control device and the light-emitting device are activated. If the target activation mode is an ultrasonic and temperature control activation mode, the temperature control device and the ultrasonic device are activated. If the target activation mode is a light, ultrasonic, and temperature control activation mode, the temperature control device, the light-emitting device, and the ultrasonic device are activated.

At block 103, when the light-emitting device is activated, irradiate the platelet-rich plasma based on at least one preset light parameter, and/or when the ultrasonic device is activated, ultrasonically treat the platelet-rich plasma based on at least one preset ultrasonic parameter, and/or when the temperature control device is activated, adjust a temperature of an environment where the platelet-rich plasma is located based on at least one preset temperature control parameter, such that platelets are activated to release growth factors and exosomes.

In one embodiment, the at least one preset light parameter includes a preset wavelength, a preset light intensity range, and a preset irradiation duration. If the target activation mode includes a light activation mode, the light-emitting device is controlled to emit light with the preset wavelength, a light intensity sensed by a light sensor of the electronic device is obtained, and if the light intensity sensed by the light sensor is out of the preset light intensity range, the wavelength of the light is adjusted until the light intensity sensed by the light sensor falls within the preset light intensity range, and the light-emitting device is controlled to irradiate the platelet-rich plasma for the preset irradiation duration.

In one embodiment, the at least one preset ultrasonic parameter includes a preset activation duration, a preset frequency, and a preset duty cycle. If the target activation mode includes an ultrasonic activation mode, the ultrasonic device is controlled to generate ultrasonic waves based on the preset frequency and the preset duty cycle, and is further controlled to guide the ultrasonic waves to propagate in the platelet-rich plasma for the preset activation duration.

In one embodiment, the at least one preset temperature control parameter includes a first preset temperature range. In all target activation modes, the temperature control device is controlled to maintain the temperature of the environment where the platelet-rich plasma is located within the first preset temperature range.

In one embodiment, the at least one preset temperature control parameter further includes a second preset temperature range and a corresponding preset duration, each temperature in the first preset temperature range is greater than each temperature in the second preset temperature range. If the target activation mode includes a temperature control activation mode, the temperature control device is controlled to maintain the temperature of the environment where the platelet-rich plasma is located within the second preset temperature range for the preset duration, and then the temperature control device is controlled to increase the temperature until the temperature reaches the first preset temperature range, and maintain the temperature of the environment where the platelet-rich plasma is located within the first preset temperature range.

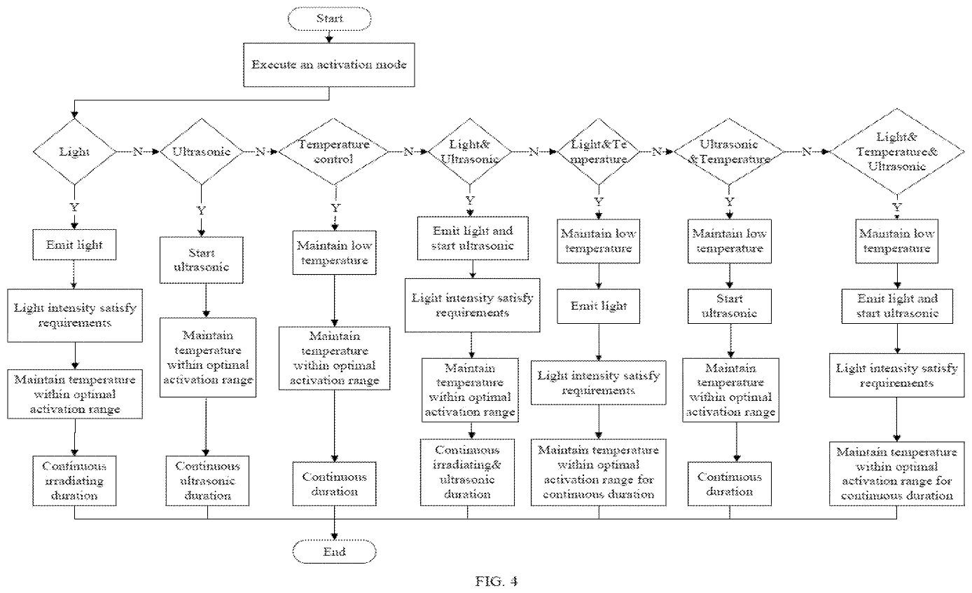

FIG. 4 illustrates a schematic view of parameter control corresponding to a target activation mode. The execution device executes the target activation mode based on a control instruction sent by the control device.

If the target activation mode is the light activation mode, the at least one preset light parameter includes a preset wavelength and a preset light intensity range. For example, the preset wavelength range is 400-1200 nm (nanometers), and the preset light intensity range is 300-500 lx (lux). The light-emitting device is controlled to emit light with the preset wavelength, the light intensity sensed by the light sensor is obtained, and it is determined whether the light intensity sensed by the light sensor is within the preset light intensity range. If the light intensity sensed by the light sensor is out of the preset light intensity range, the light is adjusted until the light intensity sensed by the light sensor falls within the preset light intensity range. Then, the temperature control device is activated to maintain the temperature of the environment where the platelet-rich plasma is located within an optimal activation range, for example, the optimal activation range is 30-37 °C. Under the preset light intensity range and the environmental temperature corresponding to the optimal activation range, the light-emitting device irradiates the platelet-rich plasma for a preset irradiation duration.

If the target activation mode is the ultrasonic activation mode, the at least one preset ultrasonic parameter includes a preset activation duration, a preset frequency, and a preset duty cycle. For example, the preset frequency range is 20 kHz-1 MHz. The ultrasonic device is controlled to generate ultrasonic waves based on the preset frequency and the preset duty cycle, and is further controlled to guide the ultrasonic waves to propagate in the platelet-rich plasma. Then, the temperature control device is activated to maintain the temperature of the environment where the platelet-rich plasma is located within an optimal activation range, for example, the optimal activation range is 30-37 °C. Under the environmental temperature corresponding to the optimal activation range, the ultrasonic device generates ultrasonic waves for the preset activation duration.

In another embodiment, the at least one preset ultrasonic parameter may further include a preset amplitude range. During the process of the ultrasonic device generating ultrasonic waves, the amplitude of the ultrasonic waves detected by the ultrasonic amplitude feedback device is obtained. If the amplitude of the ultrasonic waves is out of the preset amplitude range, the ultrasonic frequency or duty cycle is adjusted until the amplitude of the ultrasonic waves falls within the preset amplitude range.

If the target activation mode is the temperature control activation mode, the at least one preset temperature control parameter includes a number of preset temperature control stages and a preset temperature range and a temperature control duration corresponding to each preset temperature control stage. For example, the number of preset temperature control stages include two preset temperature control stages, the preset temperature range (i.e., the second preset temperature range) corresponding to the first preset temperature control stage is 4-20 °C, and the preset temperature range (i.e., the first preset temperature range) corresponding to the second preset temperature control stage is 30-37 °C. In the first preset temperature control stage, the temperature of the environment where the platelet-rich plasma is located is maintained within the preset temperature range of 4-20 °C, and the temperature control device maintains a low temperature for a first preset duration. Then, in the second preset temperature control stage, the temperature is increased by the temperature control device to fall within the optimal activation range, and maintain the temperature of the environment where the platelet-rich plasma is located within the optimal activation range (30-37 °C) for a second preset duration.

If the target activation mode is the light and ultrasonic activation mode, the at least one preset light parameter includes a preset wavelength and a preset light intensity range. For example, the preset wavelength range is 400-1200 nm, and the preset light intensity range is 300-500 lx. The at least one preset ultrasonic parameter includes a preset activation duration, a preset frequency, and a preset duty cycle. For example, the preset frequency range is 20 kHz-1 MHz. In detail, the light-emitting device is controlled to emit light with the preset wavelength, and the ultrasonic device is controlled to generate ultrasonic waves based on the preset frequency and the preset duty cycle. The light intensity sensed by the light sensor is obtained, and it is determined whether the light intensity sensed by the light sensor is within the preset light intensity range. If the light intensity sensed by the light sensor is out of the preset light intensity range, the light is adjusted until the light intensity sensed by the light sensor falls within the preset light intensity range. Then, the temperature control device is activated to maintain the temperature of the environment where the platelet-rich plasma is located within an optimal activation range, for example, the optimal activation range is 30-37 °C. Under the preset light intensity range and the environmental temperature corresponding to the optimal activation range, the light-emitting device irradiates the platelet-rich plasma for a preset irradiation duration, and the ultrasonic device generates ultrasonic waves for the preset activation duration.

If the target activation mode is the light and temperature control activation mode, the at least one preset light parameter includes a preset wavelength and a preset light intensity range. For example, the preset wavelength range is 400-1200 nm, and the preset light intensity range is 300-500 lx. The at least one preset temperature control parameter includes a number of preset temperature control stages and a temperature range and a temperature control duration corresponding to each preset temperature control stage. For example, the number of preset temperature control stages include two preset temperature control stages, the preset temperature range corresponding to the first preset temperature control stage is 4-20 °C, which is used to inhibit pre-activation, and the preset temperature range corresponding to the second preset temperature control stage is 30-37 °C, which is used to trigger controlled release and avoid premature exhaustion. In detail, in the first preset temperature control stage, the temperature control device maintains a low temperature for a first preset duration. Then, the light-emitting device is controlled to emit light with the preset wavelength. The light intensity sensed by the light sensor is obtained, and it is determined whether the light intensity sensed by the light sensor is within the preset light intensity range. If the light intensity sensed by the light sensor is out of the preset light intensity range, the light is adjusted until the light intensity sensed by the light sensor falls within the preset light intensity range. Then, in the second preset temperature control stage, the temperature is increased by the temperature control device to fall within the optimal activation range, and maintain the temperature of the environment where the platelet-rich plasma is located within the optimal activation range (30-37 °C) for a second preset duration. Under the preset light intensity range and the environmental temperature corresponding to the optimal activation range, the light-emitting device irradiates the platelet-rich plasma for a preset irradiation duration.

If the target activation mode is the light, ultrasonic, and temperature control activation mode, the at least one preset light parameter includes a preset wavelength and a preset light intensity range. For example, the preset wavelength range is 400-1200 nm, and the preset light intensity range is 300-500 lx. The at least one preset ultrasonic parameter includes a preset activation duration, a preset frequency, and a preset duty cycle. For example, the preset frequency range is 20 kHz-1 MHz. The at least one preset temperature control parameter includes a number of preset temperature control stages and a preset temperature range and temperature control duration corresponding to each preset temperature control stage. For example, the number of preset temperature control stages include two preset temperature control stages, the preset temperature range corresponding to the first preset temperature control stage is 4-20 °C, and the preset temperature range corresponding to the second preset temperature control stage is 30-37 °C.

In detail, in the first preset temperature control stage, the temperature control device maintains a low temperature for a first preset duration. Then, the light-emitting device is controlled to emit light with the preset wavelength, and the ultrasonic device is controlled to generate ultrasonic waves based on the preset frequency and the preset duty cycle. The light intensity sensed by the light sensor is obtained, and it is determined whether the light intensity sensed by the light sensor is within the preset light intensity range. If the light intensity sensed by the light sensor is out of the preset light intensity range, the light is adjusted until the light intensity sensed by the light sensor falls within the preset light intensity range. Then, in the second preset temperature control stage, the temperature is increased by the temperature control device to fall within the optimal activation range, and maintain the temperature of the environment where the platelet-rich plasma is located within the optimal activation range (30-37 °C) for a second preset duration. Under the preset light intensity range and the environmental temperature corresponding to the optimal activation range, the light-emitting device irradiates the platelet-rich plasma for a preset irradiation duration, and the ultrasonic device generates ultrasonic waves for the preset activation duration.

The embodiments of the present disclosure, based on the light activation mode, can control irradiation with different light wavelengths to activate platelets to release required growth factors in large quantities according to requirements of different patients for different growth factors. Based on the light and ultrasonic activation mode, ultrasound and light complement each other's effects, covering a wider range of growth factors and simultaneously increasing the release amount of exosomes. Based on the temperature control activation mode, by adjusting the temperature curve, it can flexibly match acute repair (rapid heating) or long-term regeneration (stepwise heating). Based on the light, ultrasonic, and temperature control activation mode, a more comprehensive and rapid activation of platelets is achieved, enabling platelets to release a large quantity of required growth factors. The embodiments of the present disclosure, by performing real-time detection and control of light intensity and temperature, can ensure safe and effective activation of platelets within an optimal range, avoiding over-activation or incomplete activation.

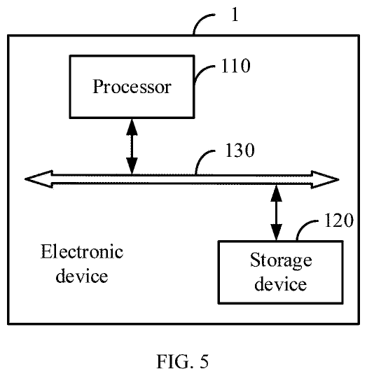

FIG. 5 illustrates a block diagram of an embodiment of an electronic device. The method for activating platelets to release growth factors and exosomes provided in this embodiment is applied to an electronic device 1, the electronic device 1 includes, but is not limited to, a processor 110 and a storage device 120 connected via a communication bus 130. FIG. 5 is merely an example of the electronic device and does not constitute a corresponding limitation. In other embodiments, the electronic device may include more components than shown.

The storage device may include one or more Random Access Memories (RAM) and one or more Non-Volatile Memories (NVM). RAM can be directly read from and written to by the processor and may be used to store an operating system, executable programs of other running programs (e.g., machine instructions), or data for users and applications. RAM may include SRAM, DRAM, SDRAM, DDR SDRAM, etc.

NVM can also store executable programs and user/application data, which can be loaded into RAM in advance for direct read/write by the processor 110. NVM may include disk storage devices and flash memory.

The storage device is configured to store one or more computer programs. The one or more computer programs are configured to be executed by the processor. The one or more computer programs include a number of instructions. When executed by the processor, the instructions implement the pre-start control method executed on the electronic device.

In other embodiments, the electronic device may also include an external memory interface for connecting external memory to expand the storage capacity of the electronic device.

The processor may include one or more processing units, e.g., an Application Processor (AP), a modem processor, a Graphics Processing Unit (GPU), an Image Signal Processor (ISP), a controller, a video codec, a Digital Signal Processor (DSP), a baseband processor, and/or a Neural-network Processing Unit (NPU). Different processing units may be independent devices or integrated into one or more processors.

The processor provides computing and control capabilities. For example, the processor is used to execute computer programs stored in the memory to implement the aforementioned pre-start control method.

The communication bus provides a channel for communication between the storage device, processor, and other components within the electronic device.

It is understood that the structure illustrated in this embodiment does not constitute a specific limitation on the electronic device. In other embodiments of the present disclosure, the electronic device may include more or fewer components than illustrated, or combine certain components, or split certain components, or have different component arrangements. The illustrated components may be implemented in hardware, software, or a combination of software and hardware.

The present disclosure further provides a computer storage medium having computer instructions stored therein. When the computer instructions are run on an electronic device, they cause the electronic device to execute the relevant method steps to implement the pre-start control method described in the above embodiments.

The present disclosure further provides a computer program product. When the computer program product runs on a computer, it causes the computer to execute the relevant steps to implement the pre-start control method described in the above embodiments.

Furthermore, an embodiment of the present disclosure provides an apparatus, which may specifically be a chip, component, or module. The apparatus includes a connected processor and memory. The memory is for storing computer-executable instructions. When the apparatus runs, the processor can execute the computer-executable instructions stored in the memory to cause the chip to perform the pre-start control method described in the various method embodiments above.

The electronic device, computer storage medium, computer program product, or chip provided in the embodiments of the present disclosure are all used to execute the corresponding methods provided above. Therefore, the beneficial effects they can achieve can be referred to the beneficial effects in the corresponding methods described above and will not be repeated here.

Through the description of the above embodiments, those skilled in the art can clearly understand that for the convenience and brevity of description, only the division of the above functional modules is used as an example. In practical applications, the above functions can be assigned to different functional modules as needed, i.e., the internal structure of the apparatus can be divided into different functional modules to complete all or part of the functions described above.

In the several embodiments provided in the present disclosure, it should be understood that the disclosed apparatus and method may be implemented in other ways. For example, the apparatus embodiments described above are merely illustrative. For instance, the division of modules or units is only a logical functional division. In actual implementation, there may be other division methods. For example, multiple units or components may be combined or integrated into another apparatus, or some features may be omitted or not executed. Further, the mutual coupling or direct coupling or communication connection shown or discussed may be indirect coupling or communication connection through some interfaces, apparatuses, or units, and may be electrical, mechanical, or otherwise.

The units described as separate components may or may not be physically separate. The components displayed as units may be one physical unit or multiple physical units, i.e., located in one place or distributed across multiple different places. Some or all of the units may be selected according to actual requirements to achieve the purposes of the embodiments.

Further, the functional units in the various embodiments of the present disclosure may be integrated into one processing unit, or each unit may exist physically separately, or two or more units may be integrated into one unit. The integrated unit may be implemented in hardware or in the form of a software functional unit.

If the integrated unit is implemented as a software functional unit and sold or used as a standalone product, it may be stored in a readable storage medium. Based on this understanding, the technical solution of the embodiments of the present disclosure, in essence or the part contributing to the prior art, or all or part of the technical solution, may be embodied in the form of a software product. The software product is stored in a storage medium and includes instructions for causing a device (which may be a microcontroller, chip, etc.) or processor to perform all or part of the steps of the methods described in the various embodiments of the present disclosure. The aforementioned storage medium includes: U disk, mobile hard disk, Read-Only Memory (ROM), Random Access Memory (RAM), magnetic disk, optical disk, or any other medium that can store program code.

It is believed that the present embodiments and their advantages will be understood from the foregoing description, and it will be apparent that various changes may be made thereto without departing from the spirit and scope of the disclosure or sacrificing all of its material advantages, the examples hereinbefore described merely being embodiments of the present disclosure.

What is claimed is:

1. A method for activating platelets to release growth factors and exosomes, applicable in an electronic device, comprising:
determining a target activation mode for platelet-rich plasma based on a target usage of the platelet-rich plasma;
activating at least one of a light-emitting device, an ultrasonic device, and a temperature control device of the electronic device based on the target activation mode;
in response to a determination that the light-emitting device is activated, irradiating the platelet-rich plasma based on at least one preset light parameter;
in response to a determination that the ultrasonic device is activated, ultrasonically treating the platelet-rich plasma based on at least one preset ultrasonic parameter; and
in response to a determination that the temperature control device is activated, adjusting a temperature of an environment where the platelet-rich plasma is located based on at least one preset temperature control parameter, and enabling platelets to be activated to release growth factors and exosomes.

2. The method according to claim 1, further comprising:
receiving the target usage of the platelet-rich plasma input by a user; and
determining the target activation mode for the platelet-rich plasma based on the target usage of the platelet-rich plasma, the target activation mode is determined from a plurality of preset activation modes, the plurality of preset activation modes comprising a light activation mode, an ultrasonic activation mode, a temperature control activation mode, a light and ultrasonic activation mode, a light and temperature control activation mode, an ultrasonic and temperature control activation mode, and a light, ultrasonic, and temperature control activation mode.

3. The method according to claim 2, wherein determining the target activation mode for the platelet-rich plasma based on the target usage of the platelet-rich plasma comprises:
determining a preset activation mode of the plurality of preset activation modes corresponding to the target usage based on a relationship between a plurality of usages and the plurality of preset activation modes and the target usage; and
determining the preset activation mode corresponding to the target usage as the target activation mode.

4. The method according to claim 2, wherein determining the target activation mode for the platelet-rich plasma based on the target usage of the platelet-rich plasma comprises:
sending the target activation mode to an interaction interface of the electronic device;
displaying the target activation mode via the interaction interface for a confirmation input on the interaction interface;
in response to a determination that the confirmation of the target activation mode is not received, receiving a preset activation mode of the plurality of preset activation modes input by the user; and
determining the preset activation mode input by the user as the target activation mode.

5. The method according to claim 1, wherein the at least one preset light parameter comprises a preset wavelength, a preset light intensity range, and a preset irradiation duration,
irradiating the platelet-rich plasma based on the at least one preset light parameter comprises:
controlling the light-emitting device to emit light with the preset wavelength;
obtaining a light intensity sensed by a light sensor of the electronic device;
in response to a determination that the light intensity sensed by the light sensor is out of the preset light intensity range, adjusting the wavelength of the light until the light intensity sensed by the light sensor is within the preset light intensity range; and
controlling the light-emitting device to irradiate the platelet-rich plasma for a preset irradiation duration.

6. The method according to claim 1, wherein the at least one preset ultrasonic parameter comprises a preset activation duration, a preset frequency, and a preset duty cycle,
ultrasonically treating the platelet-rich plasma based on the at least one preset ultrasonic parameter comprises:
controlling the ultrasonic device to generate ultrasonic waves based on the preset frequency and the preset duty cycle, controlling the ultrasonic device to guide the ultrasonic waves to propagate in the platelet-rich plasma for the preset activation duration.

7. The method according to claim 1, wherein the at least one preset temperature control parameter comprises a first preset temperature range,
adjusting the temperature of the environment where the platelet-rich plasma is located based on the at least one preset temperature control parameter comprises:
controlling the temperature control device to maintain the temperature of the environment where the platelet-rich plasma is located within the first preset temperature range.

8. The method according to claim 7, wherein the at least one preset temperature control parameter further comprises a second preset temperature range and a corresponding preset duration, each temperature in the first preset temperature range is greater than each temperature in the second preset temperature range,
in response to a determination that the target activation mode comprises a temperature control activation mode, adjusting the temperature of the environment where the platelet-rich plasma is located based on the at least one preset temperature control parameter further comprises:
controlling the temperature control device to maintain the temperature of the environment where the platelet-rich plasma is located within the second preset temperature range for the preset duration; and
controlling the temperature control device to increase the temperature, and maintain the temperature of the environment where the platelet-rich plasma is located within the first preset temperature range.

9. A electronic device comprising:
at least one processor; and
a storage device storing instructions, which when executed by the at least one processor, cause the at least one processor to:
determine a target activation mode for platelet-rich plasma based on a target usage of the platelet-rich plasma;
activate at least one of a light-emitting device, an ultrasonic device, and a temperature control device of the electronic device based on the target activation mode;
in response to a determination that the light-emitting device is activated, irradiate the platelet-rich plasma based on at least one preset light parameter;
in response to a determination that the ultrasonic device is activated, ultrasonically treat the platelet-rich plasma based on at least one preset ultrasonic parameter; and
in response to a determination that the temperature control device is activated, adjust a temperature of an environment where the platelet-rich plasma is located based on at least one preset temperature control parameter, and enable platelets to be activated to release growth factors and exosomes.

10. The electronic device according to claim 9, wherein the at least one processor is further caused to:
receive the target usage of the platelet-rich plasma input by a user; and
determine the target activation mode for the platelet-rich plasma based on the target usage of the platelet-rich plasma, wherein the target activation mode is determined from a plurality of preset activation modes, the plurality of preset activation modes comprises a light activation mode, an ultrasonic activation mode, a temperature control activation mode, a light and ultrasonic activation mode, a light and temperature control activation mode, an ultrasonic and temperature control activation mode, and a light, ultrasonic, and temperature control activation mode.

11. The electronic device according to claim 10, wherein the at least one processor is further caused to:
determine a preset activation mode of the plurality of preset activation modes corresponding to the target usage based on a relationship between a plurality of usages and the plurality of preset activation modes and the target usage; and
determine the preset activation mode corresponding to the target usage as the target activation mode.

12. The electronic device according to claim 10, wherein the at least one processor is further caused to:
send the target activation mode to an interaction interface of the electronic device;
display the target activation mode via the interaction interface for a confirmation input on the interaction interface;
in response to a determination that the confirmation of the target activation mode is not received, receiving a preset activation mode of the plurality of preset activation modes input by the user; and
determine the preset activation mode input by the user as the target activation mode.

13. The electronic device according to claim 9, wherein the at least one preset light parameter comprises a preset wavelength, a preset light intensity range, and a preset irradiation duration,
the at least one processor is further caused to:
control the light-emitting device to emit light with the preset wavelength;
obtain a light intensity sensed by a light sensor of the electronic device;
in response to a determination that the light intensity sensed by the light sensor is out of the preset light intensity range, adjust the wavelength of the light until the light intensity sensed by the light sensor is within the preset light intensity range; and
control the light-emitting device to irradiate the platelet-rich plasma for a preset irradiation duration.

14. The electronic device according to claim 9, wherein the at least one preset ultrasonic parameter comprises a preset activation duration, a preset frequency, and a preset duty cycle,
the at least one processor is further caused to:
control the ultrasonic device to generate ultrasonic waves based on the preset frequency and the preset duty cycle, and control the ultrasonic device to guide the ultrasonic waves to propagate in the platelet-rich plasma for the preset activation duration.

15. The electronic device according to claim 9, wherein the at least one preset temperature control parameter comprises a first preset temperature range,
the at least one processor is further caused to:
control the temperature control device to maintain the temperature of the environment where the platelet-rich plasma is located within the first preset temperature range.

16. The electronic device according to claim 15, wherein the at least one preset temperature control parameter further comprises a second preset temperature range and a corresponding preset duration, each temperature in the first preset temperature range is greater than each temperature in the second preset temperature range,
the at least one processor is further caused to:
control the temperature control device to maintain the temperature of the environment where the platelet-rich plasma is located within the second preset temperature range for the preset duration; and
control the temperature control device to increase the temperature, and maintain the temperature of the environment where the platelet-rich plasma is located within the first preset temperature range.

17. A non-transitory storage medium having stored thereon instructions that, when executed by a processor of an electronic device, causes the processor to perform a method for activating platelets to release growth factors and exosomes, the method comprising:
determining a target activation mode for platelet-rich plasma based on a target usage of the platelet-rich plasma;
activating at least one of a light-emitting device, an ultrasonic device, and a temperature control device of the electronic device based on the target activation mode;
in response to a determination that the light-emitting device is activated, irradiating the platelet-rich plasma based on at least one preset light parameter;
in response to a determination that the ultrasonic device is activated, ultrasonically treating the platelet-rich plasma based on at least one preset ultrasonic parameter; and
in response to a determination that the temperature control device is activated, adjusting a temperature of an environment where the platelet-rich plasma is located based on at least one preset temperature control parameter, and enabling platelets to be activated to release growth factors and exosomes.

18. The non-transitory storage medium according to claim 17, wherein the method further comprises:
receiving the target usage of the platelet-rich plasma input by a user; and
determining the target activation mode for the platelet-rich plasma based on the target usage of the platelet-rich plasma, the target activation mode being determined from a plurality of preset activation modes, the plurality of preset activation modes comprising a light activation mode, an ultrasonic activation mode, a temperature control activation mode, a light and ultrasonic activation mode, a light and temperature control activation mode, an ultrasonic and temperature control activation mode, and a light, ultrasonic, and temperature control activation mode.

19. The non-transitory storage medium according to claim 18, wherein determining the target activation mode for the platelet-rich plasma based on the target usage of the platelet-rich plasma comprises:
determining a preset activation mode of the plurality of preset activation modes corresponding to the target usage based on a relationship between a plurality of usages and the plurality of preset activation modes and the target usage; and
determining the preset activation mode corresponding to the target usage as the target activation mode.

20. The non-transitory storage medium according to claim 18, wherein determining the target activation mode for the platelet-rich plasma based on the user input of the target usage of the platelet-rich plasma further comprises:
sending the target activation mode to an interaction interface of the electronic device;
displaying the target activation mode via the interaction interface for a confirmation input on the interaction interface;
in response to a determination that the confirmation of the target activation mode is not received, receiving a preset activation mode of the plurality of preset activation modes input by the user; and
determining the preset activation mode input by the user as the target activation mode.
